(19) United States
(12) Patent Application Publication
(10) Pub. No.: US 2022/0202661 A1
(43) Pub. Date: Jun. 30, 2022

(54) (UADDP) Under Armor Deodorizer and Deodorant Pad

(71) Applicant: Leah Ayanna Brown-Johnson, Woodland Park, NJ (US)

(72) Inventor: Leah Ayanna Brown-Johnson, Woodland Park, NJ (US)

(21) Appl. No.: 17/134,370

(22) Filed: Dec. 26, 2020

(51) Int. Cl. A61K 8/02; A61Q 15/00; A61K 8/04

(52) U.S. Cl. CPC A61K 8/0208; A61K 2800/87; A61K 8/042; A61Q 15/00

(57) ABSTRACT

The (UADDP) Under Armor Deodorizer and Deodorant Pad, is a two-part system. It includes a disposable and /or a reusable deodorant pad which includes a deodorant gel insert. The deodorant pad comes in varying shapes and sizes to accommodate different armpits. The disposable pad will absorb the moisture, emit deodorant through the gel insert and is self-adhesive to the skin. It is easily removable. Both pads absorb the sweat, the deodorant provides the odor protection and the back of the pad surface traps the moisture and blocks it from staining the clothes. The reusable deodorant pad is used with a dry weave cotton material that is both breathable, and absorbs moisture. It includes a snap-able space where the deodorant insert can be placed, used, removed and then the deodorant pad can be washed.

### PREAMBLE

The purpose of this product is to respond to the markers demands for nontoxic, all-natural deodorants that absorb moisture, protect clothing, shield from embarrassing sweat accidents, and to provide a much-needed new spin on a necessary hygiene product for this generation. The Under Armor deodorizer is a two-part product: it is an underarm deodorant pad with a deodorant insert. It comes as two versions of a self-adhesive underarm pad that absorbs moisture: disposable or reusable with a breathable, washable cloth. The pad allows for the absorption of moisture to protect clothes. It is a self absorbent, all-natural, discreet underarm pad. The second part is the deodorizer insert that is provided separately for the reusable deodorant pad or is infused in the disposable pad. It allows for the body to sweat and it allows the user to avoid those embarrassing wet spots and those destructive sweat stains that damage favorite shirts while providing all day odor protection.This product also appeals to the health-conscious who prefer all-natural deodorant alternatives that actually hold odor and absorb moisture.

### CROSS REFERENCED TO RELATED APPLICATIONS

This application is not cross referenced to any other related applications.

### FEDERALLY SPONSORED RESEARCH & DEVELOPMENT

This is not applicable. This is not federally sponsored for research or development.

### DESCRIPTION OF THE FIGURES OF THE DRAWING

I, Leah Ayanna Brown-Johnson have invented a new design for an Under Armor Deodorizer and Deodorant Pad (UADDP) as set forth in the following specification:

FIG. 1 is a front perspective view of the reusable UADDP design in a soft diamond shape thereof;

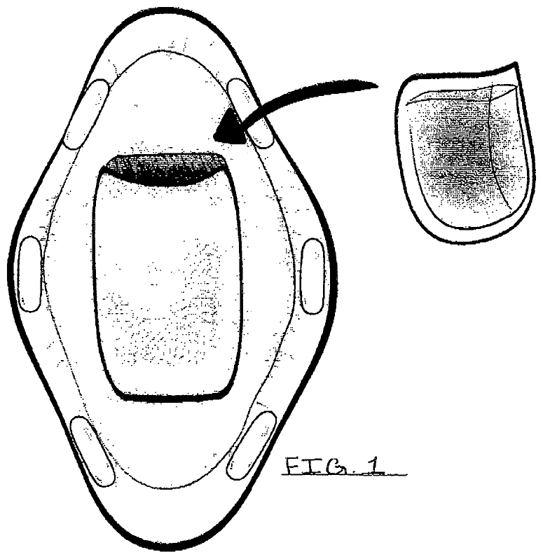

FIG. 2 is a front perspective view of the reusable UADDP in an oval shape thereof;

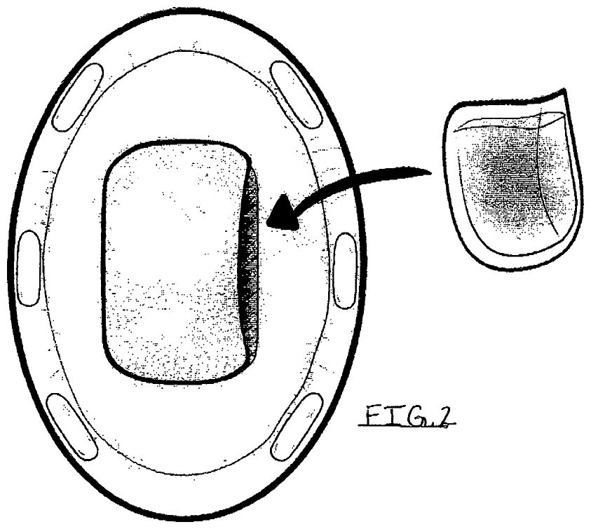

FIG. 3 is a front perspective view of the disposable UADDP in a triangular shape thereof;

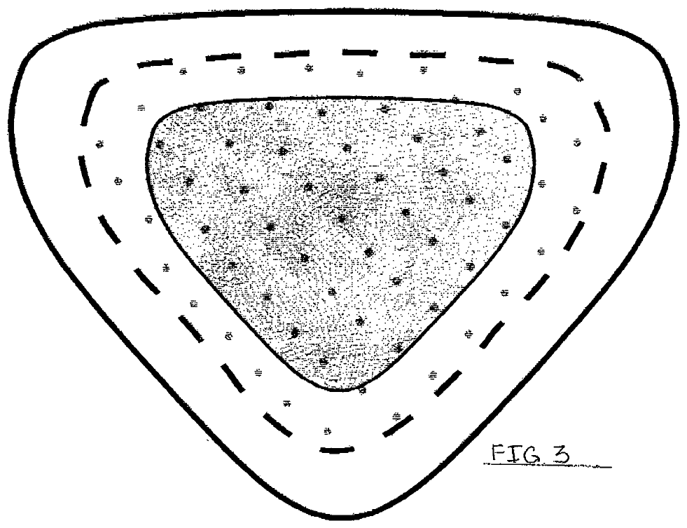

FIG. 4 is a front perspective view of the disposable UADDP in an oval shape thereof;

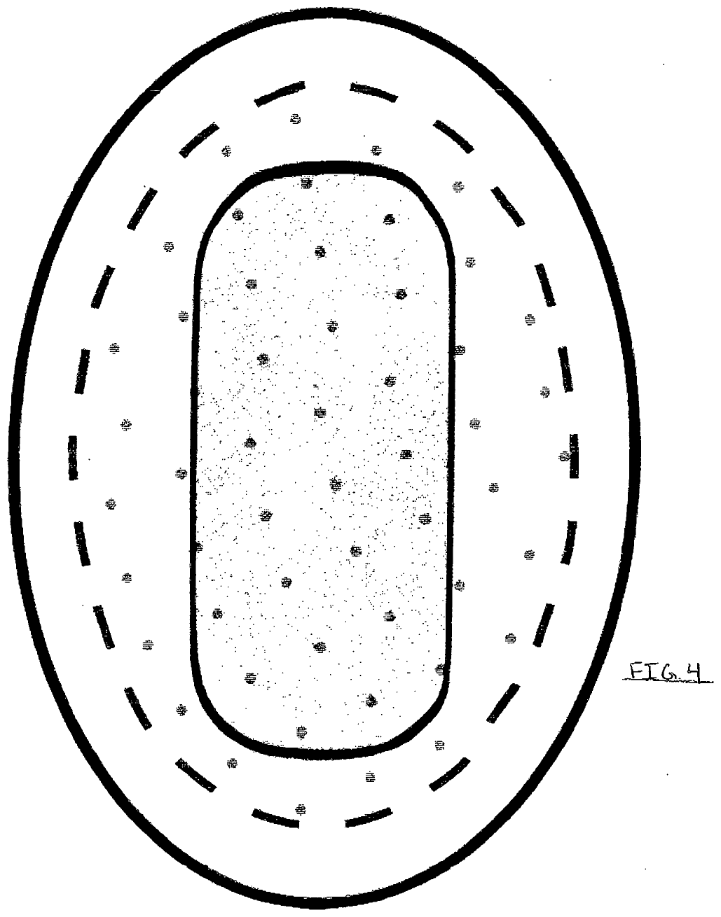

FIG. 5 is a front perspective view of the UADDP when worn under the arm thereof;

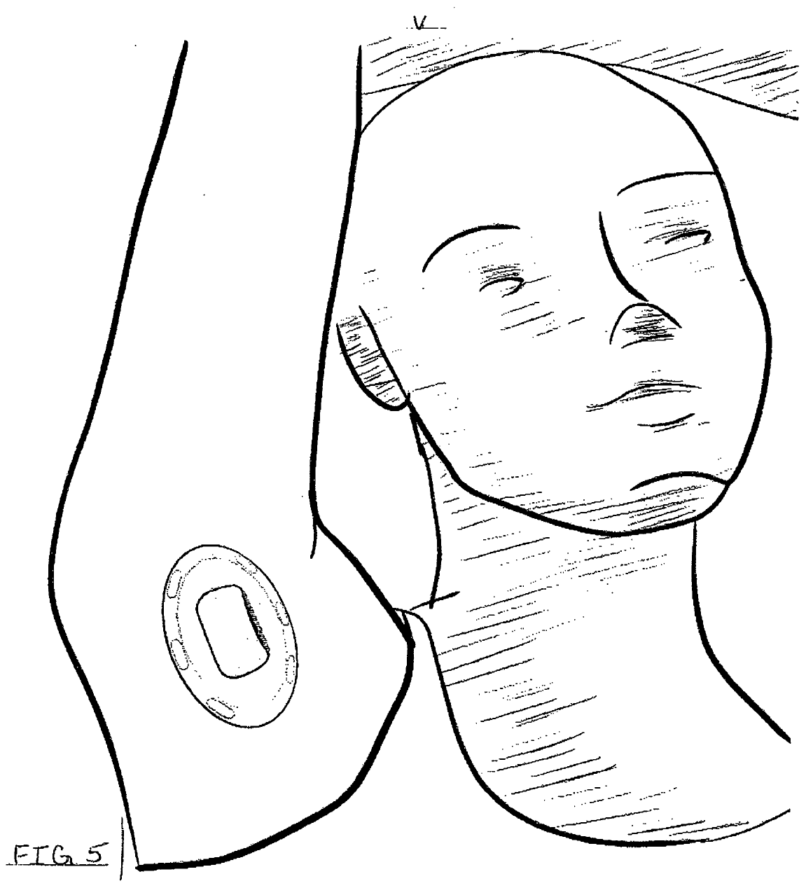

FIG. 6 is a front perspective view of the reusable UADDP with labels thereof; and

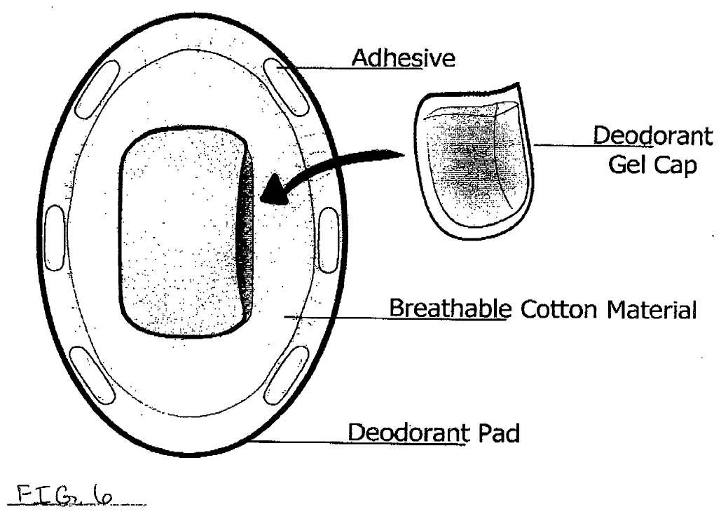

FIG. 7 is a front perspective view of the disposable UADDP with labels thereof.

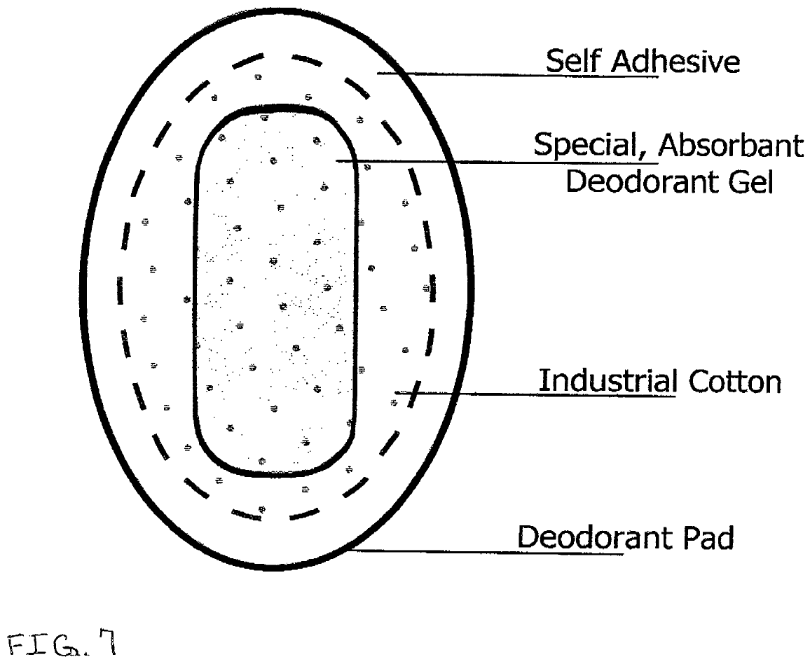

1. A personal hygiene apparatus, comprising:
a reusable or disposable pad;
with an insert for the deodorant gel with a self adhesive surrounding to secure to the body.
